(19) United States
(12) Patent Application Publication
(10) Pub. No.: US 2025/0072742 A1
(43) Pub. Date: Mar. 6, 2025

(54) INFLATABLE SPECULUM APPARATUS AND METHOD TO USE

(71) Applicants: YVONNE WOLNY, Chicago, IL (US); Martinho Pais, North Barrington, IL (US)

(72) Inventors: YVONNE WOLNY, Chicago, IL (US); Martinho Pais, North Barrington, IL (US)

(21) Appl. No.: 18/461,711

(22) Filed: Sep. 6, 2023

(51) Int. Cl. A61B 1/32; A61B 1/00; A61B 1/015; A61B 1/303

(52) U.S. Cl. CPC A61B 1/32; A61B 1/00082; A61B 1/00128; A61B 1/015; A61B 1/303

(57) ABSTRACT

Embodiments of the disclosed apparatus and method relate to a vaginal speculum used in medical office and surgical settings for an examination and treatment. The disclosed apparatus entails an inflatable vaginal speculum with features that facilitate adjustment of the diameter and the depth of penetration into the area of interest, and a camera module that provides a light source for optimal visualization of the entire anatomical area. Adjustment of the vaginal speculum's diameter and location is enabled by a method to introduce and control the amount of fluid in the apparatus to optimize the dilation and visualization of the vaginal canal. Evacuation of any refuse from the vaginal canal is enabled by using a jet or spray of fluid to irrigate and clear the surfaces of the anatomical area.

### FIELD OF THE INVENTION

The disclosed invention relates to an apparatus and method to internally traverse, dilate, illuminate, and visualize a human canal. Specifically, the apparatus relates to accessing the vaginal canal in the form of a coaxial inflatable quasi-cylindrical-annular frame arrangement henceforth called “inflatable vaginal speculum” that upon inflation with a pressurized fluid attains a cylindrical-annular balloon shape, with features that facilitate the adjustment of the diameter and the depth of penetration into the area of interest, and with minimal discomfort to the patient. An option to illuminate is integratable into the apparatus, so as to visualize both the internal side walls of the vaginal canal, and the cervix. Furthermore, a method to introduce and control the amount of fluid in the apparatus to optimize the dilation and visualization of the vaginal canal, is disclosed. Evacuation of any refuse or secretions from the vaginal canal is enabled using a jet or spray of fluid to irrigate and clear the surfaces of the anatomical area.

### BACKGROUND ART

Vaginal speculums are indispensable, and thus widely used in the field of obstetrics and gynecology for the purpose of examination and treatment. An adequate examination of the vaginal canal and the cervix requires an unobstructed visual access to the entire internal region of the anatomical area that entails the lateral vaginal walls, the vaginal fornices, and the front face of the cervix. Irrigation and removal of excess secretions in the examination area entails an important part of a successful examination and treatment in circumstances where an excessive amount of secretions is present.

The vaginal canal is partly cylindrical and partly curved in the lengthwise direction, see FIG. 1 and FIG. 2. The proximal entrance to the vaginal canal is called the vaginal introitus. At its distal end, the vaginal canal ends internally at the cervix that constitutes the proximal part of the uterus protruding into the distal end of the vaginal canal. The distal and upper part of the vaginal canal located lateral to the cervix is called the vaginal fornix and this region also needs to be taken into consideration during examination of the area by the operator-physician. Thus, any instrument that is used to enter and dilate the vaginal canal must have the capability to navigate through and accommodate to its inherent irregular geometry of the area of interest, specifically enabling visualization and access to the cervix and the vaginal fornices. In instances of altered anatomy following gynecological diseases (endometriosis, fibroids) or prior surgeries, access to the distal anatomical aspects of the vaginal canal becomes challenging, and thus requires more sophisticated devices and methods.

Currently used speculums, example see FIG. 3, comprise two blades, an upper blade and a lower blade with a handle, which when using a lever mechanism, the “thumb hinge”, allows for expanding the area of the vaginal canal in the upper and lower directions. The thumb screw facilitates holding the set position. However, in some patients, depending on their anatomy and laxity of the vaginal tissue, the lateral walls of the vaginal canal are not expanded sufficiently, resulting in the sidewalls caving in, thus obstructing the view. Further, where the patient needs gynecological procedures to be performed utilizing electrocautery or laser, the need for an unobstructed view is especially essential, demanding some means to prevent collapse of the lateral vaginal walls to enable proper visualization and to limit risk from injury due to unintended exposure of the tissue to the surgical procedures and types of energy used, viz. electrocautery, laser. Furthermore, this is also particularly important in obstetrical diagnostic-operative procedures where the tissue laxity is enhanced.

In certain circumstances where access to the area of interest, the cervix and the vaginal fornices is challenging, there is a need to navigate the anatomical area of interest by using an adjustable apparatus with regards to the length and diameter by the operator-physician. An additional limitation of present commercially used speculums is the length of the blades, which cannot be extended beyond the preset manufactured fixed length. Based on the above, there is a need for a speculum that supports a variable diameter and depth of insertion. Depending on the anatomy of the vaginal canal and the position of the cervix, a more flexible speculum would be beneficial for a thorough and safe examination and treatment. The combination of an adjustable length and diameter of the speculum with lengthwise flexibility in curvature would be ideal for multiple applications in obstetrical and gynecological examinations-procedures where an unobstructed access to the cervix is especially crucial for locating and placement of surgical instruments. Specifically, in patients with a higher Body Mass Indexes where the access might not be easily feasible using current vaginal speculums, a device enabling unencumbered access to the distal vaginal canal and cervix regions would be especially beneficial. Furthermore, an unobstructed access to the cervix is especially paramount during the more advanced gynecological robotic-laparoscopic procedures enabling adequate placement of uterine manipulators by the operator-physician.

The use of illumination is also available in certain current speculums, by configuring the speculum as a light-guide, see FIG. 4. The current design of the speculum has an upper blade and a lower blade with an attached global illumination system. Even though such a system provides general global illumination to the lateral vaginal walls and the cervix, it has no means to selectively illuminate or brighten the area of interest. In addition, the current speculum structures do not enable optimal visualization of the examination-surgical field because the lateral vaginal walls tend to collapse into the lateral internal open space between the blades when the speculum is expanded. Current general illumination systems are at fixed distance from the distal end of the speculum which is not optimized for longer and wider vaginal canals, thus decreasing the intensity of the illumination.

To limit the vaginal side-wall collapse, attempts were made to utilize plastic or elastomeric cover sheets over the entire speculum with an opening at the proximal end. Placement of a cover sheet includes an additional step over uneven speculum surfaces prior to the exam with a possibility of slippage of the cover sheet during insertion and navigation. The cover sheet also prevents visualization and illumination of the lateral vaginal walls, which constitutes an important aspect of the procedure. Even if there is a stretchable tubular coverage of the speculum, it may not allow for conformable expansion of the vaginal walls.

There is a need to have vaginal walls and cervix that are clean of secretions-refuse and also to have an unobstructed field of view while the speculum is used during examination-treatment. Hence, a feature providing irrigation and evacuation of secretions-refuse in the region of interest would be beneficial. Such a feature is not present in currently available speculums.

Therefore, there is a need for a vaginal speculum which enables the operator-physician to create an unobstructed view of the entire vaginal canal including the lateral vaginal walls, the vaginal fornices, and the cervix at the distal end. Inspection of the lateral vaginal walls is required during the insertion part of the examination process that would be facilitated by placement of a light source preferably in the proximity of the distal aspect of the vaginal speculum. The ideal speculum, on insertion into the vaginal canal, would have the capability to expand in conformity to the geometry of the vaginal canal with minimal physical discomfort to the patient. An annular-tube-like balloon arrangement is envisaged. In the deflated state, it is stiff enough to be inserted into the vaginal canal. After insertion into the vaginal canal to the appropriate depth, the annular-tube balloon is inflated thereby also expanding the vaginal canal cavity. The soft and smooth elastomeric membrane of the annular-tube balloon would facilitate comfort. Further, the use of a topical lubricant would also benefit comfort. The annular-tube balloon would have a fill port with a pressure indicator and a check-valve. The operator-physician would have the capability to use a hand-pump, compressed fluid cartridge, or other forms of fluid supply available. The use of liquids, for example saline solution, is envisaged too. The hydrostatic pressure of the liquid acts uniformly in all directions, hence enabling uniform pressure distention of the annular-tube balloon.

The operator-physician would use the fill port valve, pressure indication, and visual access of the internal aspect of the vaginal canal to regulate the level of inflation in a controlled manner. In the event, the patient is under anesthesia, this apparatus and method would be even more applicable. Furthermore, when and if the position of the speculum needs to be adjusted, the annular-tube balloon would be partially deflated to a degree necessary to reposition the speculum. Once the optimal position would be achieved, the annular-tube balloon would be reinflated to a degree to provide the desired distention of the vaginal canal. Integration of a guide beam into the inner aspect of the speculum that is protruding through the proximal end of the speculum would further facilitate means to maneuver and stabilize the apparatus during the examination or procedure process, so the operator-physician would be able to control the position of the apparatus.

In addition, features facilitating illumination of the entire vaginal canal are needed to enable a safe examination and/or surgical procedure. Finally, a means to clear and irrigate the internal vaginal surfaces of secretions-refuse is desired and is implemented.

### SUMMARY OF THE INVENTION

The new and improved apparatus facilitates the dilation of an orifice-canal in the body of a human to allow a clear illumination, visual inspection, and unobstructed physical access to the orifice-canal with instruments for visualization, inspection, treatment, and surgery.

Specifically, the current invention is directed to a coaxial quasi-cylindrical annular inflatable speculum also called “inflatable vaginal speculum” having an inflatable annular tube-like elastomeric membrane configuration, enabling expansion of the vaginal canal while conforming to the anatomy of the patient, and a camera module assembly providing an illumination feature for a better visualization of the vaginal canal during the examination process. The cross-section of the vaginal canal is of irregular quasi-cylindrical shape while in length it may follow an irregular curve. Hence, these requirements demand a device (speculum) that facilitates the dilation of the vaginal canal while automatically conforming to the allowable expandable shape of the vaginal tissue with minimum physical discomfort to the patient.

The above is configured so as to be in a deflated state to attain a diametrically smaller and compact speculum. In the deflated state, it is stiff enough to be inserted into the vaginal canal and navigate the trajectory of the same. After insertion into the vaginal canal to the appropriate depth, the inflatable vaginal speculum is gradually inflated. It will expand radially to dilate and conform to the internal geometry of the vaginal canal, thereby also expanding the vaginal canal cavity. The soft and smooth elastomeric membrane of the inflatable vaginal speculum would facilitate comfort. Further, the use of a topical lubricant would also benefit comfort. The operator-physician would have the capability to use a hand pump, compressed fluid cartridge, or other forms of fluid supply available. The hydrostatic pressure of the fluid would act uniformly in all directions, hence enabling uniform pressure to distend the inflatable vaginal speculum. Based on the pressure and visual access cues, the operator-physician would determine the desired level of distention using the check-valve arrangement.

The inflatable vaginal speculum prevents collapsing of the lateral walls of the vaginal canal into the examination-operative field enabling the operator-physician an unobstructed view of the vaginal canal area of interest. An additional benefit of the adjustable shape of the inflatable vaginal speculum includes the extended length of the speculum beyond the length of current speculums available for use. The feature is especially beneficial for patients with longer vaginal canals where access using current speculums is limited.

A further benefit of the inflatable vaginal speculum includes the capability of rotational movements to access asymmetric location of the target organ (cervix) in certain patients.

Adjustments of the diameter and the length of the inflatable vaginal speculum combined with illumination of the internal vaginal canal walls and the cervix provides the operator-physician with a much better access to the area of interest in multiple clinical settings. In order to optimize the desired diameter of the inflatable vaginal speculum, a quasi-cylindrical annular-tube structure is implemented. In addition to achieving a desired diameter, the depth of insertion can be adjusted by sliding the speculum apparatus in or out. In the event, the position of the speculum needs to be adjusted, the physician-operator may deflate the inflatable vaginal speculum, readjust the depth/direction, and then re-inflate the speculum. To further provide stability to the speculum's position in the vaginal canal during the examination or procedure, a guide beam is integrated that protrudes through the proximal end of the inflatable vaginal speculum for a distance of at least about 2 inches (5.0 cm). In the event, the speculum slips during its use, it can be stabilized by using the guide beam.

In addition to being useful in office settings, the speculum apparatus can be used in surgical settings where adequate visualization of the surgical field is especially crucial while performing minimally invasive and advanced gynecological procedures.

In the field of obstetrics, an inflatable vaginal speculum is also very useful taking into account the much looser tissue of the vaginal canal during pregnancy. To adequately visualize the cervix and the lateral vaginal walls, the speculum enables a clear view, which combined with the attached light source provides an excellent illumination of the lateral vaginal walls and the cervix. In order to follow the insertion path, an illumination device would be placed and activated during the process.

Depending on the desired distention of the vaginal canal and patient tolerance to the dilation (the patient is awake), the vaginal speculum's fluid pressure can be adjusted by the operator-physician based on a visual access to perform the procedure.

The inflatable vaginal speculum eliminates an additional step necessary in the use of certain conventional speculums of attaching a sheet-cover to facilitate a smooth insertion and to hold the lateral walls of the vaginal canal from caving into the field of view. Further, it also eliminates the discomfort when using currently commercially available metal and plastic speculums.

In addition to an unobstructed view of the area of interest, visualization of the vaginal canal and the cervix are facilitated by dual, independent high resolution digital cameras with corresponding adjustable intensity light sources. For ease of use, the speculum in its deflated state would have an attached guide beam to the internal aspect of the inflatable vaginal speculum. This design would enable visualization of the entry of the speculum including the distal aspect of the vaginal canal, the vaginal fornices, and the cervix.

For that purpose, one camera is facing the front-axial direction providing an axial view, specifically that of the cervix. The second camera is radial/side facing, providing a view of the vaginal fornices and lateral vaginal walls. Activation of the illumination assembly is useful during the insertion process to navigate and identify proper placement of the distal part of the speculum and the desired diagnostic or therapeutic procedure.

The system supports viewing the captured images of the front and lateral views via connecting over Wi-Fi, Bluetooth or wired USB, etc. to the operator-physician selectable display devices including a mobile device, computer, smart TV, or AR-VR glasses. This feature is especially useful in obstetrical and surgical settings, and any setting where documentation of the procedure is indicated. An optical and/or digital magnification of the area of interest is also supported. Finally, the system includes the option to inject fluid for the purpose of clearing refuse-secretions from the area being examined or operated on.

The method facilitates the operator-physician with the ability to dilate the area of interest, traverse, and view the vaginal canal fornices and the cervix while introducing instrumentation to perform necessary diagnostic and therapeutic procedures. Adjustments of the diameter and the depth of insertion into the vaginal canal enable procedures in challenging clinical scenarios where the region of interest is out of the range of currently available speculums. Further, the method enhances visualization of the area of interest providing illumination to the front of the cervix, the vaginal fornices, and the lateral walls of the vaginal canal followed by a capture of the images on dual high-resolution cameras. Additional features include the ability to store and print the images of the examination or procedure for further use.

### BRIEF DESCRIPTION OF THE DRAWINGS

FIG. 1 is a frontal view of the pelvic region of a female.

FIG. 2 is a side view of the pelvic region of a female.

FIG. 3 is a perspective view of a speculum with a handle and two blades.

FIG. 4 is a frontal view of a two-blade plastic speculum with an attached light source.

FIG. 5 illustrates side, axial, and perspective views of an inflated quasi-cylindrical annular elastomeric membrane with a self-sealing check-valve.

FIG. 6 illustrates side, axial, and perspective views of a frame-band structural arrangement.

FIG. 7 illustrates side, axial, and perspective views of the inflatable vaginal speculum with an external elastomeric membrane, in an inflated state.

FIG. 8 illustrates side, axial, and perspective views of the inflatable vaginal speculum with an external elastomeric membrane, in a deflated state.

FIG. 9 illustrates side, axial, and perspective views of the inflatable vaginal speculum with an internal elastomeric membrane, in an inflated state.

FIG. 10 illustrates side, axial, and perspective views of the inflatable vaginal speculum with an internal elastomeric membrane, in a deflated state.

FIG. 11 illustrates side, axial, and perspective views of the camera module.

FIG. 12 illustrates axial and perspective views of the camera module support clip.

FIG. 13 illustrates side, axial, and perspective views of the camera module assembled with two support clips.

FIG. 14 illustrates side, axial, and perspective views of the inflatable vaginal speculum apparatus with an external elastomeric membrane, in an inflated state, and a camera module attached to the guide beam with support clips.

FIG. 15 illustrates side, axial, and perspective views of the inflatable vaginal speculum apparatus with an internal elastomeric membrane, in an inflated state, and a camera module attached to the guide beam with support clips.

FIG. 16 is a flowchart of a method of assembly of the check-valve to the quasi-cylindrical annular elastomeric membrane.

FIG. 17 is a flowchart of a method of insertion of the inflatable vaginal speculum into the vaginal canal and further assembly of the camera module.

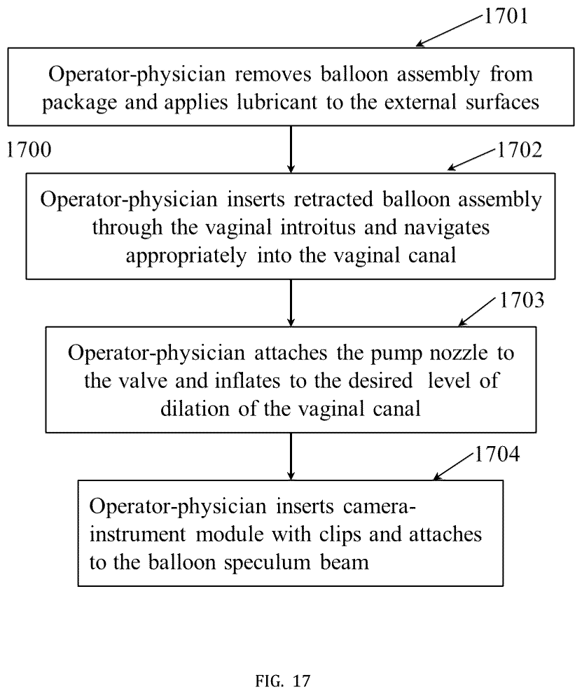

FIG. 18 is a flowchart of a method of removal of the camera module and further removal of the inflatable vaginal speculum from the vaginal canal.

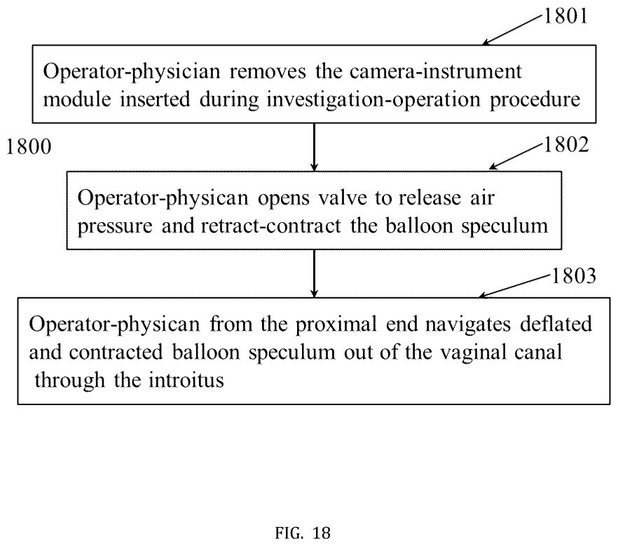

### DETAILED DESCRIPTION OF THE DRAWINGS

The present disclosure of an inflatable vaginal speculum apparatus comprising a coaxial inflatable quasi-cylindrical-annular frame arrangement herein referred to as an “inflatable vaginal speculum”, and a camera module is to be considered as an exemplification of the invention and is not intended to limit the invention by illustrations of the embodiments or their description. It should be noted that there are many variations and modifications of the apparatus and method disclosed here that would be apparent to one skilled in the art, and that are within the scope of the inventive concepts.

Embodiments of the described invention provide various novel apparatus and improvements over available speculums allowing adjustable expansion of the entire vaginal canal, visualization of the lateral walls of the vagina canal, the vaginal fornices, and the cervix. Illumination of the entire area is permitted by attachment of an integrated dual camera and a light source module to the inflatable vaginal speculum. In addition, the camera enables magnification of the area of interest, means of imaging with zoom and focus capability, means to translate the imaging module down the length of the canal in the axial direction so as to view straight down, means to rotate the imaging device to view the side walls of the vaginal canal at any axial location, light sources that illuminates in the axial and sideways directions with brightness control, and imaging module that remotes the image via Wi-Fi, BT, or wired-USB to an operator-physician selectable display device. The remote display device includes a combination of a mobile device, computer, smart TV, or AR-VR glasses.

Elimination of refuse-secretions present in the vaginal canal is effectuated by a feature supporting injection of a jet-spray of fluid, and subsequent evacuation of the refuse in the vaginal canal.

The inflatable vaginal speculum facilitates means to navigate, translate, and rotate the speculum within the vaginal canal. The speculum has structural features to actuate the level of dilation/deflation in the radial direction in conformity with the geometry of the vaginal canal. Further, it is long enough so as to be inserted into the vaginal canal with the distal end to be juxtaposed with the cervix and the proximal end extending external to the introitus sufficiently to provide means to hold by the operator-physician. The navigation of the speculum is envisaged to be a simple, single-hand operated to enable rotation and translation in a back-forth action. Integration of a guide beam with the frame band arrangement that protrudes externally through the proximal aspect of the speculum, facilitates means of control over the position of the speculum by the operator-physician. The inflatable vaginal speculum would be of different lengths to support the anatomy of the vaginal canal, which can be dependent on the age, the parity (number of deliveries) of the patient, and the type of procedure (obstetrical or gynecological).

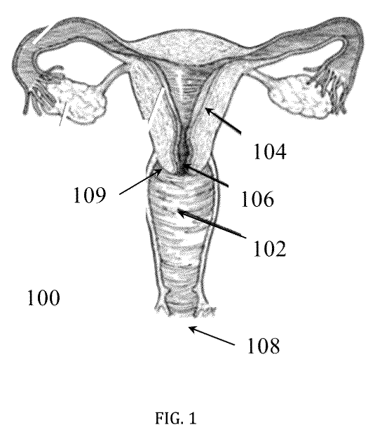

Referring to FIG. 1, it illustrates a front view of the human female pelvis 100 showing representative anatomy that includes the vaginal canal 102, the uterine corpus 104, and the cervix 106. The anatomy of the vaginal canal 102 entails a longitudinal canal extending from the opening-introitus 108 to the cervix 106. The vaginal canal 102 has a tubular shape with varying, non-uniform diameter throughout the entire length. The vaginal canal 102 is primarily composed of muscle and connective tissue. Its size, i.e., length, diameter, and thickness of wall, are highly dependent on the age, the parity, and anatomy of the patient. Generally, the introitus 108 region has a narrower diameter than the midsection and the distal and proximal end of the vaginal canal. The female cervix 106 constitutes the proximal part of the uterine corpus 104 that protrudes into the distal end of the vaginal canal 102. As illustrated in FIG. 1, the diameter of the cervix 106 is narrower than the distal end of the vaginal canal 102, which can vary depending on the age, parity, uterine pathology (uterine fibroids, endometriosis or adenomyosis), anatomical variations (anteverted or retroverted uterus), or prior surgical procedures. Upon insertion of the inflatable vaginal speculum, the diameter of the vaginal canal 102 is dilated. In addition to visualization of the lateral walls of the vaginal canal 102, the main purpose of utilizing the inflatable vaginal speculum is to visualize and have a physical instrument access to the much narrower diameter of the cervix 106. Further, there is a need to also visualize and inspect the vaginal fornices 109 to determine the anatomy and possible pathology of the region. As such, the speculum trajectory and shape need to conform to the conical shape that is wider at the proximal end of the vaginal canal (introitus) than at the distal end juxtaposed to the cervix 106.

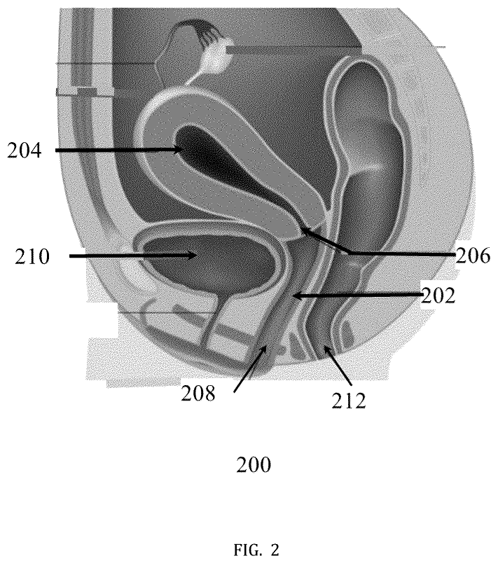

FIG. 2 represents a lateral view of the human female pelvis 200 that includes the vaginal canal 202, the uterine corpus 204, the cervix 206, the introitus 208, the bladder 210, and the rectum 212. The vaginal canal 202 is primarily composed of muscle and connective tissue. The size, i.e., length, diameter, and thickness of the vaginal wall, are highly dependent on the age and anatomy of the patient. Generally, the introitus 208 region has a narrower diameter than the midsection, the distal, and proximal end of the vaginal canal 202. The female cervix 206 constitutes the proximal part of the uterine corpus 204 that protrudes into the distal end of the vaginal canal 202. The varied length and diameter of the vaginal canal 202 demands a speculum that is not only variable in diameter and in length, but also flexible in shape to conform to the shape of the vaginal canal 202. The primary purpose of use of the speculum is not only to provide a visual access to the vaginal walls 202 and the cervix 206, but also to facilitate an unobstructed instrument access for examination and surgical procedures. Hence, there is also a need for a speculum to realize and support a desirable level of dilation of the vaginal canal 202.

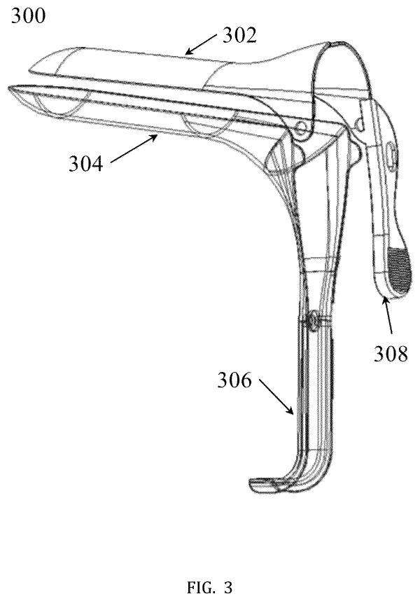

FIG. 3 represents an example “duckbill” configuration of a currently used speculum 300. Speculum 300 comprises two blades, an upper blade 302 and a lower blade 304 with a handle 306, which when using a lever mechanism in the form of a thumb operated hinge 308, allows for expanding the area of the vaginal canal in the upper and lower directions. Even though the vaginal canal is dilated in the anterior and posterior directions and physically supported, depending on the anatomy of the patient, there is a possibility of the lateral-side walls of the vaginal canal 102 to cave-fold inwards and obstruct the view. Insertion and expansion of this speculum configuration can be uncomfortable and painful to the patient, as it exerts a non-uniform and excessive pressure on the vaginal tissue at the external faces of the blades of the speculum.

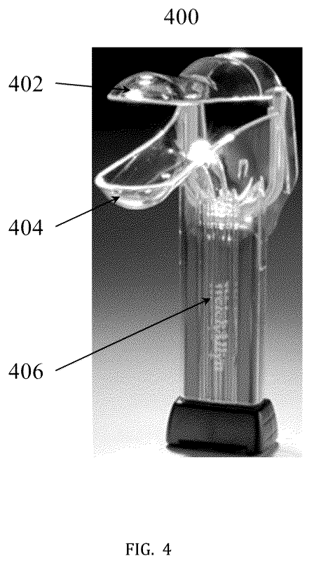

The use of illumination is also available in certain current speculums, by configuring the speculum as a light-guide, see FIG. 4. An existing design of a speculum 400 has an upper blade 402 and a lower blade 404 with an attached illumination system 406. Even though such a system provides general illumination to the lateral vaginal walls and the cervix, it has no means to selectively illuminate or brighten the area of interest. Further, the current speculum 400 does not provide means to eliminate any refuse-secretions that accumulate, especially at the distal end of the area of interest (cervix 106). Further, the location of the illumination system at the proximal end of the existing speculum 400 creates a glare from the light that is reflected proximally towards the operator-physician, and thus decreases the intensity of the illumination at the desired target.

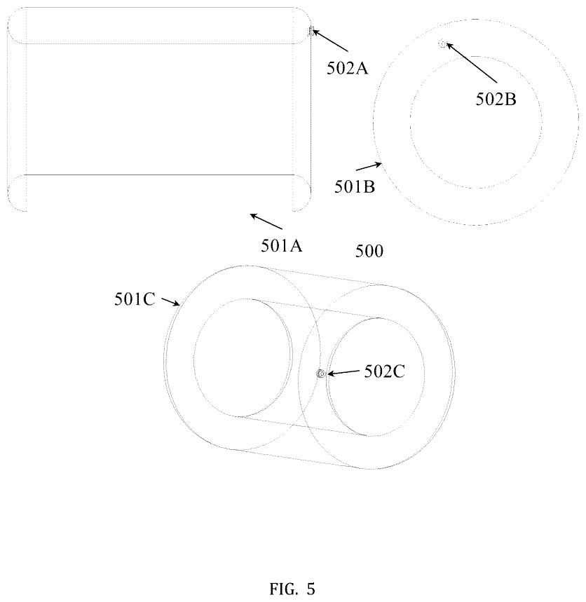

FIG. 5 represents side, axial, and perspective views of a coaxial inflatable quasi-cylindrical-annular elastomeric membrane 500, in an inflated state, with a self-sealing check-valve 502A that is installed on the proximal face of the elastomeric membrane 501A. The side view represents the elastomeric membrane, in an inflated state 501A that is effectuated by an injection of fluid through the check-valve 502A that is installed on the proximal face of the elastomeric membrane 501A. The axial view illustrates the self-sealing check-valve 502B on the proximal face of the elastomeric membrane 501B. Similarly, the perspective view illustrates the self-sealing check-valve 502C on the proximal face of the elastomeric membrane 501C. The diameter of the coaxial inflatable quasi-cylindrical-annular elastomeric membrane 501C is determined by the amount of fluid injected through the check-valve 502C by adjusting the amount of fluid using a hand-pump, compressed fluid cartridge, or other forms of pressurized fluid supply with an adjustable pressure valve available. The hydrostatic pressure of the fluid acts isotropically in all directions, hence enabling uniform pressure to distend the coaxial inflatable quasi-cylindrical-annular elastomeric membrane 500. Based on pressure and visual access cues, the operator-physician would determine the desired level of distention using the adjustable pressure valve arrangement. A class of material exists that have elastic-elastomeric properties, implying that when the pressurized fluid is released from the coaxial inflatable quasi-cylindrical-annular elastomeric membrane 500, the apparatus will deflate resulting in a decreased diameter for an ease of retraction of the speculum from the vaginal canal 102. Given that the coaxial inflatable quasi-cylindrical-annular elastomeric membrane is expandable through the use of fluid pressure, there is also a need for a structural feature to limit the geometry to an annular configuration.

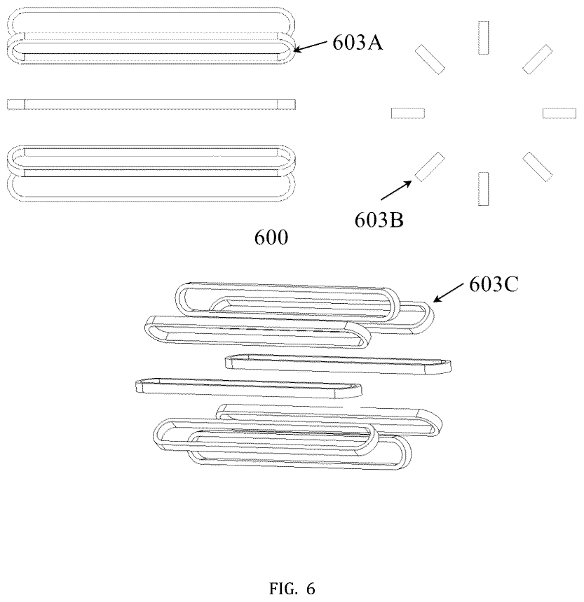

FIG. 6 represents side, axial, and perspective views of a frame-band arrangement comprising a multiplicity of circumferentially distributed axially oriented bands, 600. The frame-band 603A-C is made of flexible yet stiff material, such as plastic or metal. On inflation, it maintains its size and shape. The number of the frame-bands 603A-C are set to a minimum (at least) of three depending on the desired diameter of dilation of the vaginal canal, in an expanded state. 603A-C illustrates eight bands configured circumferentially with an annular height of about 10 mm. Therefore, in the deflated state, the minimum diameter of the speculum would be about 20 mm. The length of the frame-band is envisaged to be about 150 mm (6.0 inches), but it does not preclude shorter or longer arrangements, as demanded by the patient's anatomy.

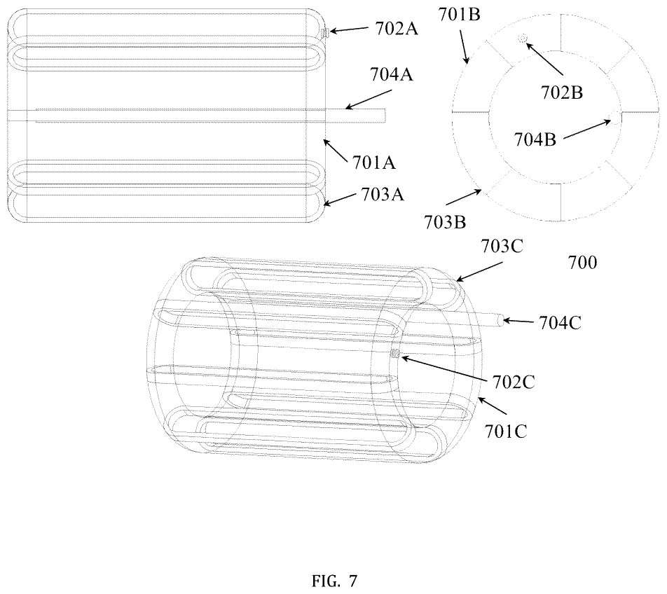

FIG. 7 illustrates side, axial, and perspective views of the inflatable vaginal speculum 700 in the inflated configuration, comprising the elastomeric membrane of FIG. 501A-C represented here as 701A-C, connected externally to the lengthwise oriented, circumferentially distributed frame-band arrangement of FIG. 603A-C represented here as 703A-C. A check-valve 702A-C is installed on the proximal face of the elastomeric membrane 701A-C to allow connection to a source of pressurized fluid that upon injection would inflate the elastomeric membrane 701A-C. The stiffer, lengthwise oriented, circumferentially distributed frame-band arrangement 703A-C facilitates maintaining the expanding elastomeric membrane 701A-C, in an annular shape. As illustrated in 700, one or more, lengthwise oriented guide beam(s) 704A-C are attached on the internal aspect to one or more of the lengthwise oriented, circumferentially distributed frame-bands 703A-C. The guide beam(s) 704A-C is (are) designed longer so as to protrude out on the proximal side about 50 mm (2.0 inches). The guide beam(s) 704A-C serve(s) the purpose to control the position and stability of the inflatable vaginal speculum 700 in the patient's vaginal canal. Further, it (they) facilitates(s) the capture of the camera to the inflatable vaginal speculum. In the event, the position of the inflatable vaginal speculum needs to be adjusted, the physician-operator can deflate, reposition, and inflate the inflatable vaginal speculum again. The length of the guide beam 704A-C is envisioned to be the same regardless of the size of the inflatable vaginal speculum, as it also provides a rail-like guide for the camera module.

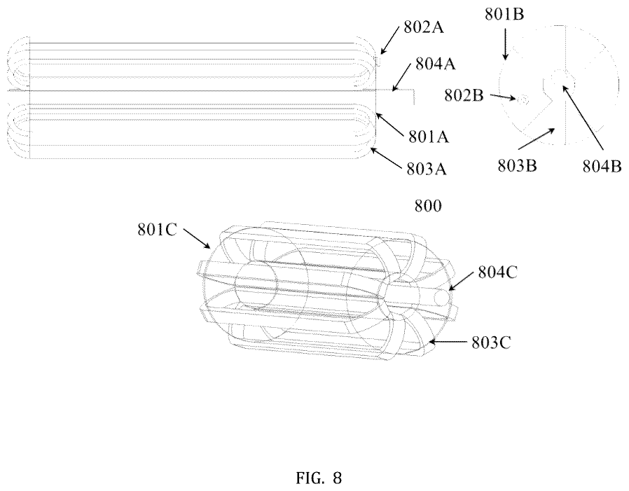

The vaginal canal 102, is a muscular quasi-cylindrical tissue which is highly elastic, and in a contracted state, under normal conditions. To introduce the speculum into the introitus 108 the inflatable vaginal speculum 600 needs to be also apriori deflated. FIG. 8 represents side, axial, and perspective views of the inflatable vaginal speculum 700, in a deflated configuration 800, comprising a lengthwise oriented frame band arrangement 803A-C and an elastomeric membrane 801A-C connected externally to the circumferentially distributed axially oriented bands 803A-C, and a check-valve 802A-C that is installed on the proximal face of the elastomeric membrane 801A-C, and lengthwise oriented guide beam(s) 804A-C running axially connected on the internal aspect to one of the circumferentially distributed axially oriented bands 803A-C. The height of each band 803A-C is approximately 10 mm. As illustrated in 800, the frame band arrangement in the deflated state will have a minimum diameter equivalent to twice the height of the lengthwise oriented, circumferentially distributed frame bands 803A-C plus the diameter(s) of the lengthwise oriented beam 804A-C for a total of about 25 mm (1.0 inch). The guide beam(s) 804A-C is (are) protruding beyond the proximal end of the inflatable vaginal speculum 800 for a distance of about 50 mm (2 inches) to facilitate insertion and position of the inflatable vaginal speculum 800 in the vaginal canal 102. The inflatable vaginal speculum 800, in the deflated state, is designed to be stiff enough to facilitate an insertion, orientation, and location in the vaginal canal 102.

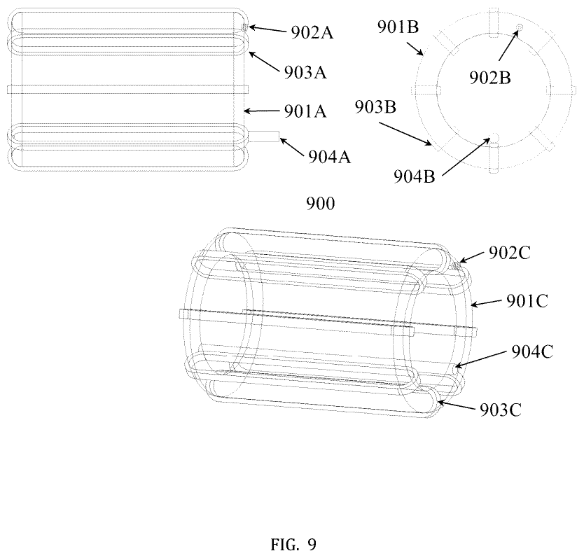

FIG. 9 represents side, axial, and perspective views of an alternative embodiment of the inflatable vaginal speculum described in FIG. 7. In this embodiment 900, the elastomeric membrane 901A-C is attached internally to the frame band arrangement 903A-C. The elastomeric membrane of FIG. 501A-C represented here as 901A-C, is connected internally to the lengthwise oriented, circumferentially distributed frame-band arrangement of FIG. 601A-C represented here as 903A-C. A check-valve 902A-C is installed on the proximal face of the elastomeric membrane 901A-C to allow connection to a source of pressurized fluid that upon injection would inflate the elastomeric membrane 901A-C. The stiffer, lengthwise oriented, circumferentially distributed frame band arrangement 903A-C facilitates maintaining the expanding elastomeric membrane 901A-C, in an annular shape. As illustrated in 900, one or more, lengthwise oriented guide beam(s) 904A-C are attached on the internal aspect to one or more of the lengthwise oriented, circumferentially distributed frame bands 903A-C. The guide beam(s) 904A-C is (are) designed longer so as to protrude out on the proximal side about 50 mm (2.0 inches). The guide beam(s) 904A-C serve(s) the purpose to control the position and stability of the inflatable vaginal speculum 900 in the patient's vaginal canal. Further it (they) facilitates(s) the capture of the camera to the speculum. In the event, the position of the inflatable vaginal speculum needs to be adjusted, the physician-operator can deflate, reposition, and inflate the inflatable vaginal speculum again. The length of the guide beam 904A-C is envisioned to be the same regardless of the size of the inflatable vaginal speculum, as it also provides a rail-like support for the camera module.

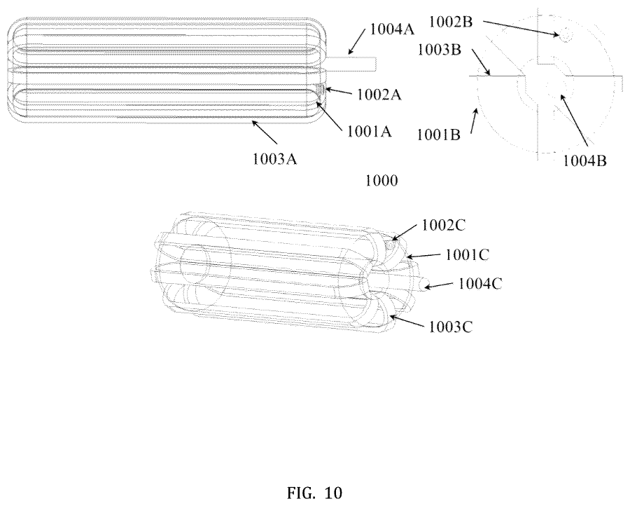

The vaginal canal 102, is a muscular quasi-cylindrical tissue which is highly elastic, and in a contracted state under normal conditions. To introduce the speculum into the introitus 108 the inflatable vaginal speculum 600 needs to be also apriori deflated. FIG. 10 represents side, axial, and perspective views of the inflatable vaginal speculum 900, in the deflated configuration 1000, comprising a lengthwise oriented frame band arrangement 1003A-C and an elastomeric membrane 1001A-C connected internally to the circumferentially distributed axially oriented bands 1003A-C, and a check-valve 1002A-C that is installed on the proximal face of the elastomeric membrane 1001A-C, and a lengthwise oriented guide beam 1004A-C running axially connected on the internal aspect to one of the circumferentially distributed axially oriented bands 1003A-C. The height of each band 1003A-C is approximately 10 mm. As illustrated in 800, the frame band arrangement in the deflated state will have a minimum diameter equivalent to twice the height of the lengthwise oriented, circumferentially distributed frame-bands 1003A-C plus the diameter(s) of the lengthwise oriented beam 1004A-C for a total of about 25 mm (1.0 inch). The guide beam 1004A-C is protruding beyond the proximal end of the coaxial inflatable quasi-cylindrical-annular frame arrangement 1000 for a distance of about 50 mm (2.0 inches) to facilitate insertion and position of the speculum on the internal aspect in the vaginal canal. The internal aspect 1000 of the speculum, in the deflated state, is designed to be stiff enough to facilitate its insertion, orientation, and location into the vaginal canal.

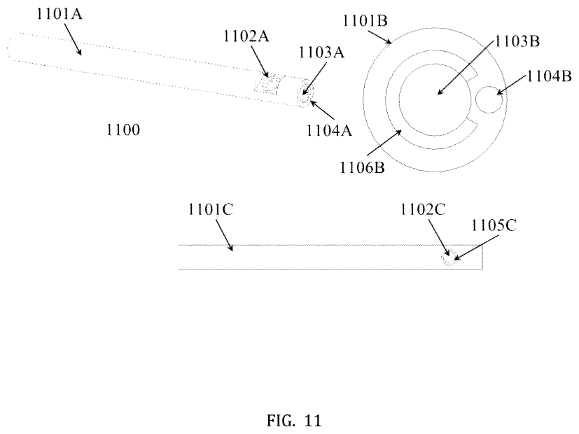

FIG. 11 illustrates side, axial, and perspective views of a camera module 1100. A housing 1101A-C encompasses the dual cameras, dual light sources, and the jet/spray. The visualization of the vaginal canal 102 is facilitated by dual high resolution digital cameras. The front camera 1103A-B provides a view directly in front and axially. The side camera 1102A-C provides a view radially or sideways, i.e., to the lateral walls of the vaginal canal 102. Dedicated adjustable intensity light sources, side 1105C and front 1106B facilitate illumination of the lateral and front regions of the vaginal canal 102. The lighting features 1105C and 1106B provide a sufficient illumination to allow for the ease of view with the camera of the extended region internal to the vaginal canal 102. The lighting feature comprises light sources being either or combination of LEDs, laser, incandescent, or fiber optic remotely piped in, where the light intensity is adjustable. The camera module assembly supports viewing the captured images of the front and the lateral views via connecting over Wi-Fi, Bluetooth, wired USB, etc. to the operator-physician selectable display devices including a mobile device, computer, smart TV, or AR-VR glasses. This feature is especially useful in obstetrical and surgical settings, and any setting where documentation of the procedure is indicated. An optical or digital magnification of the area of interest is also supported. The system of optics, lenses, optical fibers being in-situ within or integrated within the camera module 1100. Further, the camera module 1100 has features 1104 to introduce a jet/spray of fluid to cleanse-push away or irrigate any refuse-secretions present on the vaginal canal 102 and the cervix 106 surfaces.

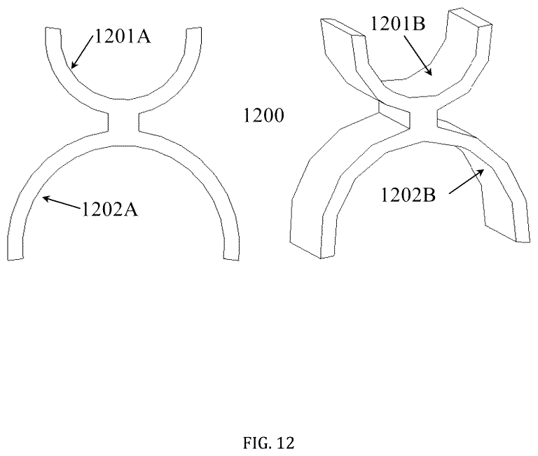

FIG. 12 illustrates side and perspective views of a camera module-instrument support clip 1200. One clasp 1201A-B of the support clip attaches to the guide beam in the inflatable vaginal speculum. The second clasp 1202A-B of the support clip attaches and holds on to the camera module. As shown in FIG. 12, at least two or more support clips are envisioned depending on the length of the speculum required for the patient.

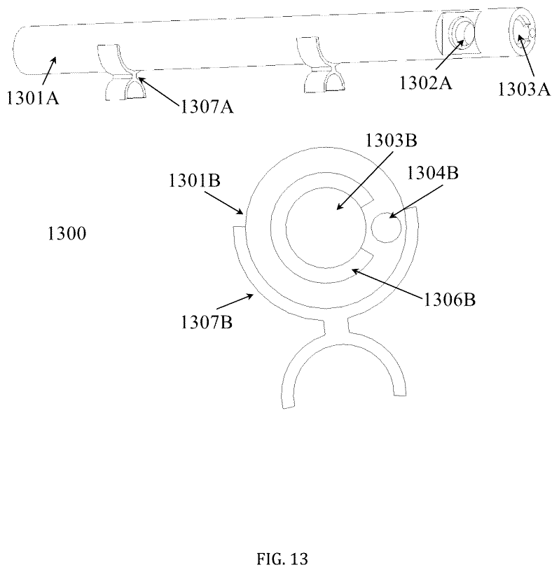

FIG. 13 illustrates an axial and perspective views of the assembly of the camera module with two support clips 1300. The camera module 1301A-B is attached to two clips 1307A-B. Further, the clips 1307A-B are arranged in line to facilitate attachment to the guide beam in the inflatable vaginal speculum. The camera module assembly supports viewing the captured images of the front and lateral views via connecting over Wi-Fi, Bluetooth, wired USB, etc. to the operator-physician selectable display devices including a mobile device, computer, smart TV, or AR-VR glasses. This feature is especially useful in obstetrical and surgical settings and any setting where documentation of the procedure is indicated. An optical or digital magnification of the area of interest is also supported. The system of optics, lenses, optical fibers being in-situ within, or integrated within the camera module 1300. Further, the camera module 1300 has features 1304B to introduce a jet/spray of fluid to cleanse-push away or irrigate any refuse-secretions present on the vaginal canal 102 and cervix 106 surfaces.

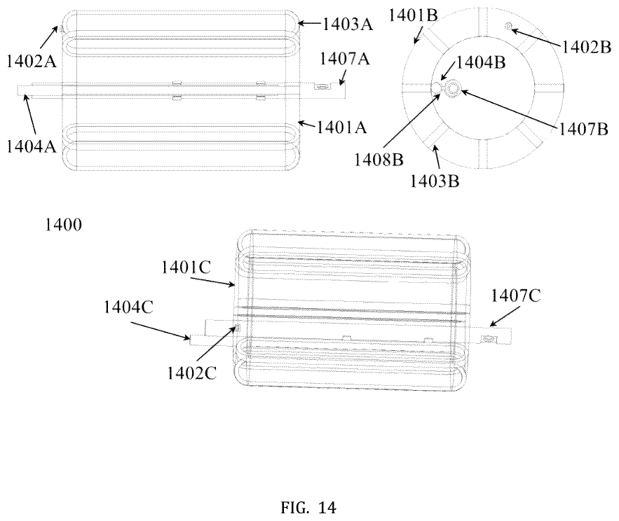

FIG. 14 represents side, axial and, perspective views of the inflatable vaginal speculum 1400, in the inflated configuration, comprising the elastomeric membrane of FIG. 501A-C, represented here as 1401A-C that is connected externally to the lengthwise oriented, circumferentially distributed frame-band arrangement of FIG. 603A-C, represented here as 1403A-C. A check-valve 1402A-C is installed on the proximal face of the elastomeric membrane 1401A-C to allow connection to a source of pressurized fluid that upon injection would inflate the elastomeric membrane 1401A-C. The stiffer, lengthwise oriented, circumferentially distributed frame-band arrangement 1403A-C facilitates maintaining the expanding elastomeric membrane 1401A-C, in an annular shape. As illustrated in 1400, one or more lengthwise oriented guide beam(s) 1404A-C are attached on the internal aspect to one or more of the lengthwise oriented, circumferentially distributed frame-bands 1403A-C. The guide beam(s) 1404A-C is (are) designed longer so as to protrude out on the proximal side about 50 mm (2.0 inches). The guide beam(s) 1404A-C serve(s) the purpose to control the position and stability of the coaxial inflatable quasi-cylindrical-annular frame arrangement 1400 in the patient's vaginal canal. Further it (they) facilitates(s) the capture of the camera module 1407A-C using the clips 1408B to the inflatable vaginal speculum. In the event, the position of the inflatable vaginal speculum needs to be adjusted, the physician-operator can deflate, reposition, and inflate the inflatable vaginal speculum again.

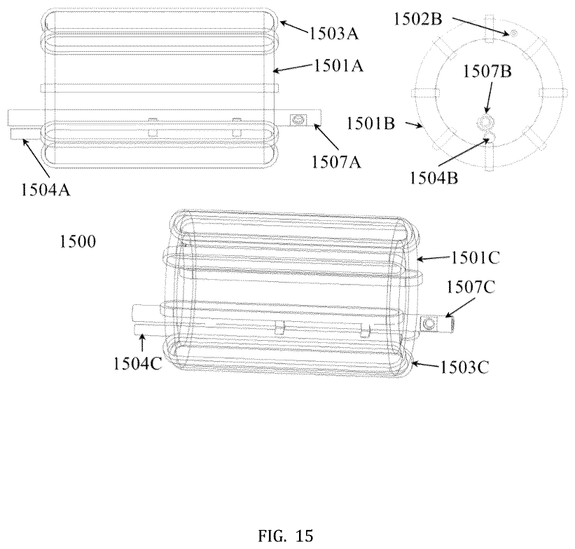

FIG. 15 represents side, axial and, perspective views of the inflatable vaginal speculum 1500 in the inflated configuration, comprising the elastomeric membrane of FIG. 501A-C, represented here as 1501A-C, connected internally to the lengthwise oriented, circumferentially distributed frame-band arrangement of FIG. 603A-C, represented here as 1503A-C. A check-valve 1502A-C is installed on the proximal face of the elastomeric membrane 1501A-C to allow connection to a source of pressurized fluid that upon injection, would inflate the elastomeric membrane 1501A-C. The stiffer, lengthwise oriented, circumferentially distributed frame-band arrangement 1503A-C facilitates maintaining the expanding elastomeric membrane 1501A-C in an annular shape. As illustrated in 1500, one or more, lengthwise oriented guide beam(s) 1504A-C are attached on the internal aspect to one or more of the lengthwise oriented, circumferentially distributed frame-bands 1503A-C. The guide beam(s) 1504A-C is (are) designed longer so as to protrude out on the proximal side about 50 mm (2.0 inches). The beam(s) 1504A-C serve(s) the purpose to control the position and stability of the coaxial inflatable quasi-cylindrical-annular frame arrangement 1500 in the patient's vaginal canal 102. Further it (they) facilitates(s) the capture of the camera module 1507A-C using clips to the inflatable vaginal speculum. In the event, the position of the inflatable vaginal speculum needs to be adjusted, the physician-operator can deflate, reposition, and inflate the apparatus again.

In the above description of exemplary embodiments of the frame-bands of rectangular geometries, are presented to illustrate feasibility. It is understood that other beam geometries may also be utilized without departing from the spirit or scope of the present disclosure. The description is, therefore, not to be taken in a limiting sense.

It is intended that the geometries used will impose minimum impact on the tissues of the vaginal canal 102, partly by proper surface treatments, such as polished and lubricated. Further, geometries are selected to spread the distention load so as to reduce pressure concentration on the tissues.

Those of ordinary skill in the art will appreciate that the hardware components may vary and are not intended to be exhaustive, but rather are representative to highlight essential components that are utilized to implement aspects of the described embodiments. For example, other devices-components may be used in addition to or in place of the hardware and/or firmware depicted. The example is not meant to imply architectural or other limitations with respect to the presently described embodiments and/or the general invention.

In the above description of exemplary embodiments of the disclosure, specific exemplary embodiments in which the various aspects of the disclosure may be practiced are described in sufficient detail to enable those skilled in the art to practice the invention, and it is to be understood that other embodiments may be utilized and that logical, architectural, programmatic, mechanical, electrical, and other changes may be made without departing from the spirit or scope of the present disclosure. The description is, therefore, not to be taken in a limiting sense.

It is understood that the use of specific component and/or apparatus/device/module names, such as those described herein, are for example only and not meant to imply any limitations on the described embodiments. Thus, each term utilized herein is to be given its broadest interpretation given the context in which that term is utilized.

### METHOD OF INVENTION

The vaginal canal 102 in its normal unexcited state is almost a retracted-collapsed wall of muscle tissue. In reality, it is an expandable quasi-cylindrical tubular shell, which with appropriate and careful physical intervention, can be expanded. Hence, there is an opportunity to insert a speculum into the vaginal canal 102 in a retracted-contracted state and after introduction cause it to expand so as to dilate the tissue of the vaginal canal walls.

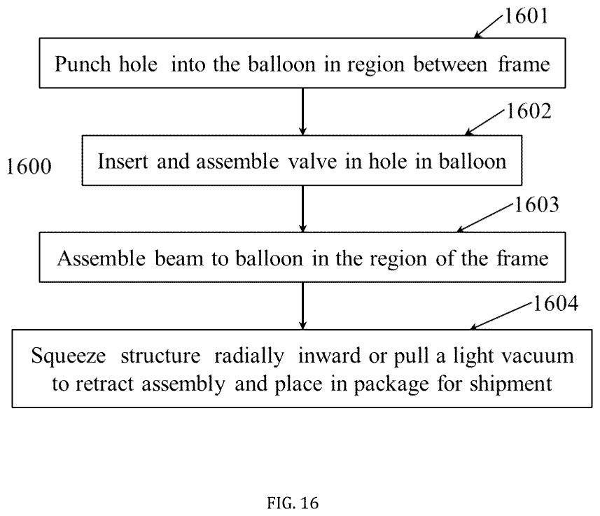

Referring to FIG. 16, the following method 1600 is proposed to assemble the inflatable vaginal speculum. It is envisaged that at the manufacturing facility, the coaxial inflatable quasi-cylindrical-annular frame arrangement will be fitted with a valve. The process entails punching a hole 1601 in the elastomeric membrane between any two of the frame-bands and then inserting the valve 1602. A guide beam will then be affixed 1603 to the internal aspect of the frame-band arrangement using an adhesive or other means of adhering. Finally, the inflatable vaginal speculum will be partly evacuated 1604 to deflate and retract the assembly and packaged for shipment to the clinics for the operator-physician use.

In the event of a vaginal examination-procedure, the operator-physician would prior to insertion of the contracted-retracted arrangement 800/1000, remove from the packaging 1604, and lubricate 1701 the external surfaces of the inflatable vaginal speculum appropriately to facilitate ease of insertion into the vaginal canal 102. Upon insertion 1702 through the introitus 108 and navigating to an adequate position, the operator-physician attaches the pump nozzle to the valve and inflates 1703 to the desired level of dilation of the vaginal canal.

The operator-physician installs two or more clips 1704 on the camera module 1100. This assembly 1300 is then inserted into the expanded inflatable vaginal speculum and clipped onto the guide beam.

On completion of the medical procedures, the operator-physician would remove instrument modules 1801 by unclipping the camera and clip assembly 1300 from the guide beam and then removing the camera module from the proximal end of the inflated inflatable vaginal speculum.

On the completion of the medical procedures, the operator-physician would deflate-contract 1802 the inflatable vaginal speculum by releasing the pressurized fluid out of the valve. Then, the operator-physician would navigate 1803 the deflated inflatable vaginal speculum in the outward proximal direction and exit it out of the introitus 108.

In the above description of exemplary embodiments of the disclosure, specific exemplary embodiments in which the various aspects of the disclosure may be practiced are described in sufficient detail to enable those skilled in the art to practice the invention, and it is to be understood that other embodiments may be utilized and that logical, architectural, programmatic, mechanical, electrical, and other changes may be made without departing from the spirit or scope of the present disclosure. The description is, therefore, not to be taken in a limiting sense.

It is understood that the use of specific component and/or apparatus/device/module names, such as those described herein, are for example only and not meant to imply any limitations on the described embodiments. Thus, each term utilized herein is to be given its broadest interpretation given the context in which that term is utilized.

What is claimed is:

1. An inflatable vaginal speculum apparatus, comprising:
an inflatable vaginal speculum, comprising an elastomeric membrane having a proximal end and a distal end, and a self-sealing check-valve that is installed on the proximal face of elastomeric membrane, and a frame band arrangement having a multiplicity of circumferentially distributed axially oriented bands, wherein the frame band arrangement is attached to the elastomeric membrane, and upon injection of fluid and expansion of the elastomeric membrane, a cylindrical-annular balloon is formed to conform to the anatomy of the vaginal canal, injection of fluid into the elastomeric membrane is effectuated through the self-sealing check-valve, and a guide beam connected to one of the circumferentially distributed axially oriented bands within the frame band arrangement; and
a camera module assembly comprising a housing and two cameras, a front camera capturing the front aspect of the area of interest, and a second camera capturing the lateral aspect of the area of interest, wherein the camera module assembly is connected to the guide beam that is attached to one of the bands in the cylindrical annular frame band arrangement to provide stability during the examination process, and a support clip(s) that facilitate attachment of the camera module to the guide beam through a clasp(s) of the support clip(s).

2. The inflatable vaginal speculum apparatus as in claim 1, wherein the frame band arrangement comprises at least three rectangular-elliptical bands circumferentially distributed and axially oriented.

3. The inflatable vaginal speculum apparatus as in claim 1, wherein the inflatable vaginal speculum has a proximal end and a distal end, the proximal end being at the introitus of the vaginal canal, and the distal end being at the cervix.

4. The inflatable vaginal speculum apparatus as in claim 1, wherein the elastomeric membrane is connected externally to the frame band arrangement, and thus creating a closed cylindrical-annular balloon configuration.

5. The inflatable vaginal speculum apparatus as in claim 1, wherein the elastomeric membrane is connected internally to the frame band arrangement, and thus creating a closed cylindrical-annular balloon configuration.

6. The inflatable vaginal speculum apparatus as in claim 1, wherein the elastomeric membrane is made of an impermeable, stretchable elastomeric material enabling inflation and subsequent expansion of the inflatable vaginal speculum to a desired state.

7. The inflatable vaginal speculum apparatus as in claim 1, wherein the elastomeric membrane has a proximal end and a distal end, wherein the check valve is installed on the proximal face of the elastomeric membrane to regulate the level of inflation and pressure.

8. The inflatable vaginal speculum apparatus as in claim 1, wherein the guide beam is oriented axially and is protruding on the proximal end of the inflatable vaginal speculum for at least two inches to provide means to handle the insertion, orientation, and removal of the vaginal speculum.

9. The inflatable vaginal speculum apparatus as in claim 1, wherein the guide beam is connected to the internal aspect of the inflatable vaginal speculum.

10. The inflatable vaginal speculum apparatus as in claim 1, in which the fluid source with a pressure relief valve and variable flow valve, is a hand or portable pump or other fluid supply such as a compressed fluid source including a cartridge or building supply wherein the pressure relief valve controls the internal pressure, and thus the level of inflation of the inflatable vaginal speculum.

11. The inflatable vaginal speculum apparatus as in claim 1, wherein the support clip(s) comprise two clasps, wherein one clasp is attached to the camera module, and the other clasp is attached to the guide beam.

12. The inflatable vaginal speculum apparatus as in claim 1, wherein the front camera capturing the front aspect of the area of interest, is configured on the distal end of the camera module assembly, and the second camera capturing the lateral aspect of the area of interest is configured on the lateral side of the distal end of the camera module.

13. The inflatable vaginal speculum apparatus as in claim 1, wherein the inflatable vaginal speculum is disposable.

14. A method for use in performing a medical procedure using an inflatable vaginal speculum apparatus comprising an inflatable vaginal speculum to dilate the vaginal canal in conformity to its shape, and a camera module to facilitate gynecological and obstetrical procedures, comprising the steps of:
providing the inflatable vaginal speculum apparatus comprising the inflatable vaginal speculum in the deflated state to the physician-operator;
applying lubricant to the external surface of the inflatable vaginal speculum;
inserting the inflatable vaginal speculum in the deflated state into the vaginal canal;
connecting a fluid source to the check-valve on the inflatable vaginal speculum;
inflating gradually an elastomeric membrane of the inflatable vaginal speculum by injecting fluid;
using a pressure relief check-valve to adjust the level of inflation of the inflatable vaginal speculum;
attaching the camera module to the clips;
attaching the clips to the guide beam within the inflatable vaginal speculum;
adjusting the camera module so as to facilitate optimal visualization of the inflatable vaginal speculum region of interest;
performing procedures on the inflatable vaginal speculum region of interest by the operator-physician;
detaching the clips from the guide beam in the inflatable vaginal speculum upon completion of the examination or procedure;
removing the camera module from the inflatable vaginal speculum through the introitus;
deflating the inflatable vaginal speculum;
removing the inflatable vaginal speculum from the vaginal canal.

15. The method as in claim 14, wherein the introducing step includes providing the inflatable vaginal speculum comprising materials with sufficient strength to maintain the vaginal canal open upon insertion of the inflatable vaginal speculum, in the inflated state.

16. The method as in claim 14, wherein upon initial inflation of the inflatable vaginal speculum, repositioning of the inflatable vaginal speculum is effectuated by partial deflating the inflatable vaginal speculum and re-inflating the inflatable vaginal speculum again once the desired position is achieved.

17. The method as in claim 14, wherein the position and orientation of the camera module on the guide beam is adjusted depending on the visualization of the region of interest by the operator-physician.

18. The method as in claim 14, wherein the fluid source comprises a pressure relief valve and variable flow valve with a hand or portable pump or other fluid supply such as a compressed fluid source including a cartridge or building supply.

19. The method as in claim 14, wherein the pressure relief valve regulates the level of inflation/deflation of the inflatable vaginal speculum.
